(19) United States
(12) Patent Application Publication
(10) Pub. No.: US 2025/0364130 A1
(43) Pub. Date: Nov. 27, 2025

(54) PROVIDING INTERACTIVE INSTRUCTIONS FOR MEDICAL APPARATUS

(71) Applicant: Boston Scientific Scimed Inc., Maple Grove, MN (US)

(72) Inventors: Narin Anderson, Shakopee, MN (US); Keith Ervin Robertson, JR., St. Michael, MN (US); Amit Rauniyar, Maple Grove, MN (US); Kirk K. Krona, Maple Grove, MN (US)

(21) Appl. No.: 19/210,392

(22) Filed: May 16, 2025

Related U.S. Application Data
(60) Provisional application No. 63/650,165, filed on May 21, 2024.

(51) Int. Cl. G16H 40/63; G06F 16/3329; G06F 16/38; G10L 13/08

(52) U.S. Cl. CPC G16H 40/63; G06F 16/33295; G06F 16/381; G10L 13/086

(57) ABSTRACT

At least some embodiments of the present disclosure are directed to systems and methods for providing interactive instructions for a medical device. In some embodiments, a method includes receiving information associated with the medical device, identifying device parameters of the medical device, causing to display a representation of the medical device associated with the device parameters of the medical device, receiving a user query related to the representation of the medical device, identifying a language of the user query, generating a device prompt based at least in part on the user query and the device parameters, and generating a query response in the identified language by applying a machine learning model, and causing to deliver the query response in the identified language.

### CROSS REFERENCE TO RELATED APPLICATION

This application claims priority to Provisional Application No. 63/650,165, filed May 21, 2024, which is herein incorporated by reference in its entirety.

### TECHNICAL FIELD

The present disclosure relates to systems and methods for providing instructions for a medical apparatus or device. More specifically, the present disclosure relates to systems and methods for providing interactive instructions for a medical apparatus or device.

### BACKGROUND

Commercialized medical devices generally provide electronic or are packaged with physical Instructions for Use (IFU) documentation. An IFU may include device information such as, for example, a device function, a maintenance status, a safety precaution, a troubleshooting of the medical device, etc. An IFU may provide an overview of the medical device, its intended use, and any important background information. It may also highlight safety information, including potential hazards, contraindications, and precautions to be taken during use. It may offer step-by-step guidance on how to assemble, operate, and maintain the medical device, for example, including diagrams or illustrations for clarity. It may offer recommendations for proper storage conditions and handling procedures to maintain the medical device integrity. An IFU may include technical details about the medical device, such as dimensions, materials, and compatibility with other equipment. It may also provide contact information for the manufacturer or distributor, including customer support and service resources.

An IFU for a medical device may be presented in various formats to accommodate different user preferences and technological capabilities. An IFU may include a printed manual, a digital manual, online documentation, and the like. A physical IFU may be vulnerable to misplacement and may potentially lead to incorrect usage due to misinterpretation of the content contained in the IFU and/or by a user failing to quickly locate and follow the guidance that is contained in the IFU.

### SUMMARY

As recited in examples, Example 1 is a method of providing interactive instructions for a medical device. The method includes receiving information associated with the medical device; identifying one or more device parameters of the medical device; causing to display a representation of the medical device associated with the one or more device parameters of the medical device; receiving a user query related to the representation of the medical device; identifying a language of the user query; generating a device prompt based at least in part on the user query and the one or more device parameters; generating a query response by applying a machine learning model to the device prompt and a data repository including information associated with the medical device, the query response being in the identified language; and causing to deliver the query response in the identified language.

Example 2 is the method of Example 1, wherein the receiving information associated with the medical device comprises receiving at least one selected from a group consisting of a quick response (QR) code, a Uniform Resource Locator (URL), a near-field-communication (NFC) tag, a radio-frequency identification (RFID) tag, and a product image.

Example 3 is the method of Example 1 or 2, further comprising retrieving the one or more device parameters of the medical device from the data repository.

Example 4 is the method of any one of Examples 1-3, wherein the receiving a user query further comprises receiving a vocalized query.

Example 5 is the method of Example 4, further comprising converting the vocalized query to a transcribed text.

Example 6 is the method of any one of Examples 1-5, wherein the generating a device prompt further comprises determining whether the user query meets one or more criteria.

Example 7 is the method of Example 6, wherein when the user query does not meet the one or more criteria, the generating a query response further comprises generating contextual information of the medical device from the data repository.

Example 8 is the method of any one of Examples 1-7, wherein the applying a machine learning model comprises generating an improved query response for the user query using the user query and the query response.

Example 9 is the method of any one of Examples 1-8, wherein the generating a query response further comprises providing one or more query response constraints.

Example 10 is the method of any one of Examples 1-9, wherein the generating a query response further comprises adjusting a content generation parameter.

Example 11 is the method of Example 10, wherein the query response generated based on the content generation parameter corresponds to a device content in the data repository, and the content generation parameter is adjusted such that a difference between the query response and the device content in the data repository is not greater than a predetermined level.

Example 12 is the method of Example 11, wherein the content generation parameter is adjusted to be lower than a predetermined threshold to control a coherency between the query response and the device content in the data repository.

Example 13 is the method of any one of Examples 1-12, further comprising converting the query response to a voice response in the identified language before delivering the query response.

Example 14 is a system of providing interactive instructions for a medical device. The system includes one or more memories having instructions stored thereon; and one or more processors configured to execute the instructions and perform operations including: receiving information associated with the medical device; identifying one or more device parameters of the medical device; causing to display a representation of the medical device associated with the one or more device parameters of the medical device; receiving a user query related to the representation of the medical device; identifying a language of the user query; generating a device prompt based at least in part on the user query and the one or more device parameters; generating a query response by applying a machine learning model to the device prompt and a data repository including information associated with the medical device, the query response being in the identified language; and causing to deliver the query response in the identified language.

Example 15 is the system of Example 14, wherein the operations further comprise receiving at least one selected from a group consisting of a quick response (QR) code, a Uniform Resource Locator (URL), a near-field-communication (NFC) tag, a radio-frequency identification (RFID) tag, and a product image.

Example 16 is the system of Example 14 or 15, wherein the operations further comprise converting a vocalized query to a transcribed text.

Example 17 is the system of any one of Examples 14-16, wherein the operations further comprise determining whether the user query meets one or more criteria, and when the user query does not meet the one or more criteria, generating contextual information of the medical device from the data repository.

Example 18 is the system of any one of Examples 14-17, wherein the operations further comprise adjusting a content generation parameter to meet one or more query response constraints.

Example 19 is the system of Example 18, wherein the query response generated based on the content generation parameter corresponds to a device content in the data repository, and the content generation parameter is adjusted such that a difference between the query response and the device content in the data repository is not greater than a predetermined level.

Example 20 is the system of any one of Examples 14-19, wherein the operations further comprise converting the query response to a voice response in the identified language before delivering the query response.

Example 21 is a system of providing interactive instructions for a medical device. The system includes one or more memories having instructions stored thereon; and one or more processors configured to execute the instructions and perform operations. The operations include receiving information associated with the medical device; identifying one or more device parameters of the medical device; causing to display a representation of the medical device associated with the one or more device parameters of the medical device; receiving a user query related to the representation of the medical device; identifying a language of the user query; generating a device prompt based at least in part on the user query and the one or more device parameters; generating a query response by applying a machine learning model to the device prompt and a data repository including information associated with the medical device, the query response being in the identified language; and causing to deliver the query response in the identified language.

Example 22 is the system of Example 21, wherein the operations further comprise receiving at least one selected from a group consisting of a quick response (QR) code, a Uniform Resource Locator (URL), a near-field-communication (NFC) tag, a radio-frequency identification (RFID) tag, and a product image.

Example 23 is the system of Example 21 or 22, wherein the operations further comprise receiving a vocalized query.

Example 24 is the system of any one of Examples 21-23, wherein the operations further comprise converting the vocalized query to a transcribed text.

Example 25 is the system of any one of Examples 21-24, wherein the operations further comprise determining whether the user query meets one or more criteria.

Example 26 is the system of Example 25, wherein when the user query does not meet the one or more criteria, generating contextual information of the medical device from the data repository.

Example 27 is the system of any one of Examples 21-26, wherein the operations further comprise providing one or more query response constraints to generate the query response.

Example 28 is the system of Example 27, wherein the operations further comprise adjusting a content generation parameter to meet the one or more query response constraints.

Example 29 is the system of Example 28, wherein the query response generated based on the content generation parameter corresponds to a device content in the data repository.

Example 30 is the system of Example 28 or 29, wherein the content generation parameter is adjusted such that a difference between the query response and the device content in the data repository is not greater than a predetermined level.

Example 31 is the system of Example 29 or 30, wherein the content generation parameter is adjusted to be lower than a predetermined threshold to control a coherency between the query response and the device content in the data repository.

Example 32 is the system of any one of Examples 21-31, wherein the operations further comprise converting the query response to a voice response in the identified language before delivering the query response.

Example 33 is the system of any one of Examples 21-32, wherein the operations further comprise retrieving the one or more device parameters of the medical device from the data repository.

Example 34 is the system of any one of Examples 21-33, wherein the applying a machine learning model comprises generating an improved query response for the user query using the user query and the query response.

Example 35 is the system of any one of Examples 21-34, wherein the machine learning model comprises a large language model (LLM).

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a system for providing interactive instructions for a medical device, in accordance with embodiments of the subject matter of the disclosure.

FIG. 2 depicts an illustrative block diagram for an Instructions for Use (IFU) system, in accordance with embodiments of the subject matter of the disclosure.

FIG. 3A is an illustrative example of a user interface, in accordance with embodiments of the subject matter of the disclosure.

FIG. 3B is an illustrative example of a user interface, in accordance with embodiments of the subject matter of the disclosure.

FIG. 4 is an example flow diagram depicting an illustrative method of providing multi-language interactive instructions for a medical device, in accordance with some embodiments of the present disclosure.

FIG. 5 is an example flow diagram depicting an illustrative method of providing multi-language, speech-to-speech (STS) interactive instructions for a medical device, in accordance with some embodiments of the present disclosure.

FIG. 6 is an example block diagram depicting an illustrative method of providing multi-language, speech-to-speech (STS) interactive instructions for a user to use a medical device, in accordance with some embodiments of the present disclosure

FIG. 7 is a simplified block diagram of a computing device, with which aspects of the present disclosure may be practiced.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

As the terms are used herein with respect to measurements (e.g., dimensions, characteristics, attributes, components, etc.), and ranges thereof, of tangible things (e.g., products, inventory, etc.) and/or intangible things (e.g., data, electronic representations of currency, accounts, information, portions of things (e.g., percentages, fractions), calculations, data models, dynamic system models, algorithms, parameters, etc.), “about” and “approximately” may be used, interchangeably, to refer to a measurement that includes the stated measurement and that also includes any measurements that are reasonably close to the stated measurement, but that may differ by a reasonably small amount such as will be understood, and readily ascertained, by individuals having ordinary skill in the relevant arts to be attributable to measurement error; differences in measurement and/or manufacturing equipment calibration; human error in reading and/or setting measurements; adjustments made to optimize performance and/or structural parameters in view of other measurements (e.g., measurements associated with other things); particular implementation scenarios; imprecise adjustment and/or manipulation of things, settings, and/or measurements by a person, a computing device, and/or a machine; system tolerances; control loops; machine-learning; foreseeable variations (e.g., statistically insignificant variations, chaotic variations, system and/or model instabilities, etc.); preferences; and/or the like.

Although illustrative methods may be represented by one or more drawings (e.g., flow diagrams, communication flows, etc.), the drawings should not be interpreted as implying any requirement of, or particular order among or between, various steps disclosed herein. However, certain some embodiments may require certain steps and/or certain orders between certain steps, as may be explicitly described herein and/or as may be understood from the nature of the steps themselves (e.g., the performance of some steps may depend on the outcome of a previous step). Additionally, a “set,” “subset,” or “group” of items (e.g., inputs, algorithms, data values, etc.) may include one or more items, and, similarly, a subset or subgroup of items may include one or more items. A “plurality” means more than one.

As used herein, the term “based on” is not meant to be restrictive, but rather indicates that a determination, identification, prediction, calculation, and/or the like, is performed by using, at least, the term following “based on” as an input. For example, predicting an outcome based on a particular piece of information may additionally, or alternatively, base the same determination on another piece of information.

The present disclosure describes systems and methods for providing interactive instructions for a medical apparatus or device. More specifically, some embodiments of the present disclosure relate to systems and methods for providing multi-language, speech-to-speech interactive instructions for a medical apparatus or device. Some embodiments of the present disclosure provide an IFU system or platform having an enhanced accessibility, efficiency, and inclusivity regarding the delivery of instructions for medical devices. Some embodiments of the present disclosure provide an IFU system or platform which allows users (e.g., physicians) worldwide to access critical, operational information regarding the medical device in a language they can understand via speech.

According to some embodiments, systems and methods for providing interactive instructions described herein use one or more computing models. In certain embodiments, a model, also referred to as a computing model, includes a model to process data. A model includes, for example, an artificial intelligence (AI) model, a machine learning (ML) model, a deep learning (DL) model, an image processing model, an algorithm, a rule, other computing models, and/or a combination thereof.

In some embodiments, a generative AI (artificial intelligence) model includes training data embedded in the model. In certain embodiments, a generative AI model is a type of AI model that can be used to produce various type of content, such as text, images, videos, audio, 3D (three-dimensional) data, 3D models, and/or the like. In some embodiments, a language model or a large language model (LLM), which is a type of generative AI model, includes content and training data embedded in the model.

In some embodiments, a machine learning (ML) model is a language model (“LM”) that may include an algorithm, rule, model, and/or other programmatic instructions that can predict the probability of a sequence of words. In some embodiments, a language model may, given a starting text string (e.g., one or more words), predict the next word in the sequence. In certain embodiments, a language model may calculate the probability of different word combinations based on the patterns learned during training (based on a set of text data from books, articles, websites, audio files, etc.). In some embodiments, a language model may generate many combinations of one or more next words (and/or sentences) that are coherent and contextually relevant. In certain embodiments, a language model can be an advanced artificial intelligence algorithm that has been trained to understand, generate, and manipulate language. In some embodiments, a language model can be useful for natural language processing, including receiving natural language prompts and providing natural language responses based on the text on which the model is trained. In certain embodiments, a language model may include an n-gram, exponential, positional, neural network, and/or other type of model.

In certain embodiments, the machine learning model is a large language model (LLM), which was trained on a larger data set and has a larger number of parameters (e.g., billions of parameters) compared to a regular language model. In certain embodiments, an LLM can understand more complex textual inputs and generate more coherent responses due to its extensive training. In certain embodiments, an LLM can use a transformer architecture that is a deep learning architecture using an attention mechanism (e.g., which inputs deserve more attention than others in certain cases). In some embodiments, a language model includes an autoregressive language model, such as a Generative Pre-trained Transformer 3 (GPT-3) model, a GPT 3.5-turbo model, a Claude model, a command-xlang model, a bidirectional encoder representation from transformers (BERT) model, a pathways language model (PaLM) 2, and/or the like. A prompt can be provided for processing by the LLM, which thus generates a response, a recommendation, or a content accordingly.

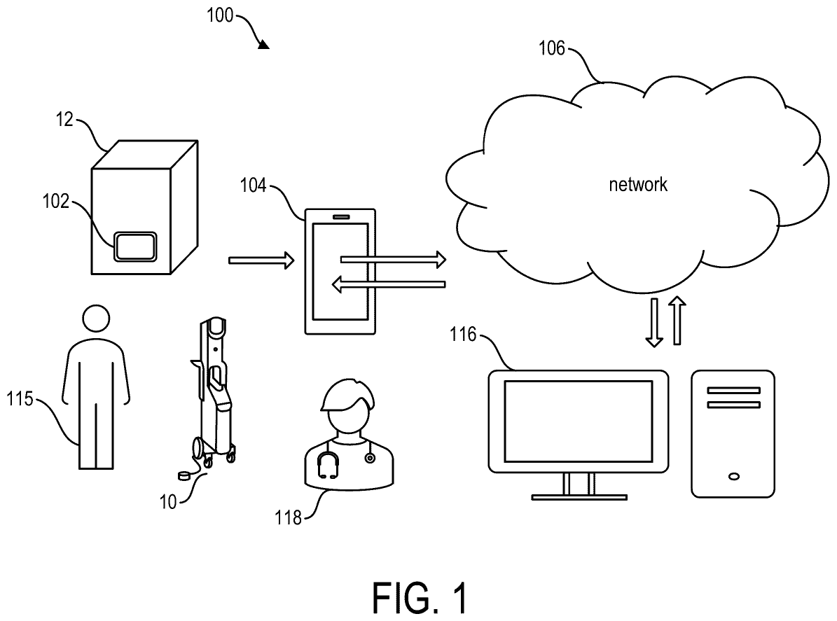

FIG. 1 illustrates an Instructions for Use (IFU) system or platform 100 for providing interactive instructions for a medical device 10, in accordance with embodiments of the subject matter of the disclosure. In some embodiments, the medical device 10 has a packaging 12, where a product label 102 is provided on the packaging 12 and/or on the medical device 10. The product label 102 can include, for example, a quick response (QR) code, a Uniform Resource Locator (URL), a shortened URL, a smart tag (e.g., a near-field-communication or NFC tag), a radio-frequency identification (RFID) tag, and the like. It is to be understood that the product label 102 may be provided in other suitable formats or manners.

The IFU system or platform 100 can be accessed by a mobile device 104 and/or a computing device 116 (e.g., a client device such as a laptop, a mobile phone, a desktop computer, and the like) by a user (e.g., a physician) 118 at a clinic or lab. The mobile device 104 and the computing device 116 can communicate data with each other over a communication network 106. The user 118 can detect the product label 102 by using a detecting device of the mobile device 104 (e.g., a smartphone, a tablet, a QR reader device, an NFC-enabled device, and the like) to obtain information associated with the medical device 10. It is to be understood that the detecting device may be a separate device functionally connected to the mobile device 104. In some embodiments, augmented reality (AR) techniques can be applied to recognize the medical device 10 or specific part(s) of the medical device 10 visually through a smartphone or AR glasses, which can then display interactive instructions directly overlaid on the real-world image.

With the detection of the medical device 10, the system or platform 100 can be initiated to provide interactive instructions to the user 118 regarding the medical device 10 and/or applying the medical device 10 to a patient 115. In an embodiment, the system or platform 100 can provide multi-language, speech-to-speech (STS) interactive instructions for the medical device 10.

According to some embodiments, the system or platform 100 can include one or more memories having instructions stored thereon, and one or more processors configured to execute the instructions and perform operations. In some embodiments, the operations can include receiving information associated with the medical device, identifying one or more device parameters of the medical device, causing to display a representation of the medical device associated with the one or more device parameters of the medical device, receiving a user query related to the representation of the medical device, identifying a language of the user query, generating a device prompt based at least in part on the user query and the one or more device parameters, generating a query response by applying a machine learning model to the device prompt and a data repository including information associated with the medical device, the query response being in the identified language, and causing to deliver the query response in the identified language.

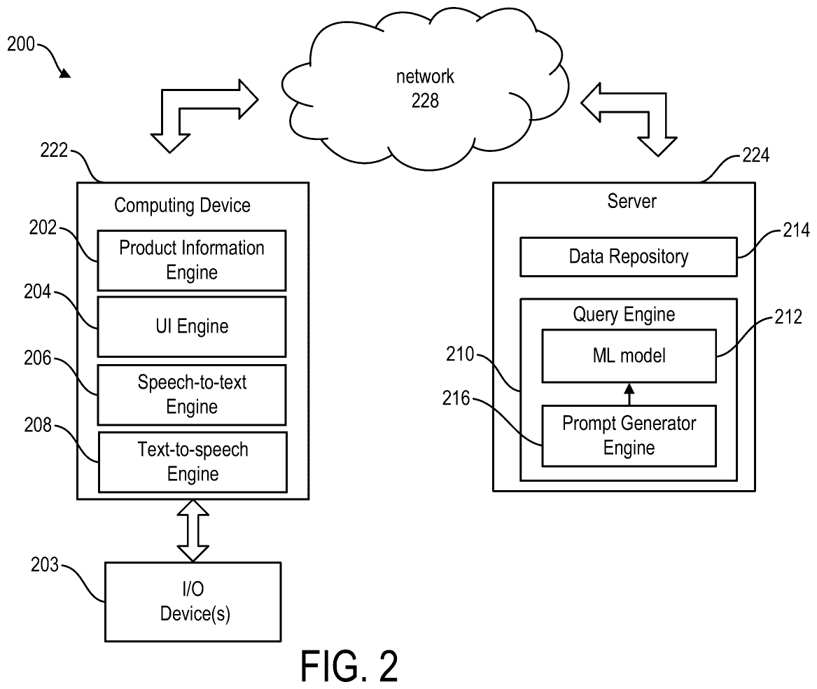

FIG. 2 depicts an illustrative block diagram for an Instructions for Use (IFU) platform or system 200, in accordance with embodiments of the subject matter of the disclosure. According to some embodiments, the system 200 includes a computing device 222 and a server 224 connected by a communication network 228.

In certain embodiments, the computing device 222 includes a product information engine 202 configured to receive information associated with the medical device from one or more input/output devices 203 to identify one or more parameters of the medical device. In an embodiment, the input/output devices 203 (e.g., a camera of a mobile device) can detect a quick response (QR) code associated with the medical device. For example, the QR code can be provided on a product packaging of the medical device. In an embodiment, the input/output devices 203 (e.g., a camera of a mobile device) can detect a shortened Uniform Resource Locator (URL) which may be printed on a product packaging of the medical device. In an embodiment, the input/output devices 203 (e.g., an input device of a mobile device) can receive the shortened URL manually typed by a user. In an embodiment, the input/output devices 203 (e.g., an NFC-enabled device) can detect a smart tag which may be provided on a product packaging of the medical device.

In some embodiments, the product information engine 202 can receive and process the detected information from the input/output devices 203 to identify one or more device parameters of the medical device such as for example, name, serial number, model number, relevant identifying information, and the like. In some embodiments, the computing device 222 further includes a user interface engine 204 which can instruct an input/output device 203 (e.g., a display) to display a representation of the medical device associated with the one or more device parameters of the medical device.

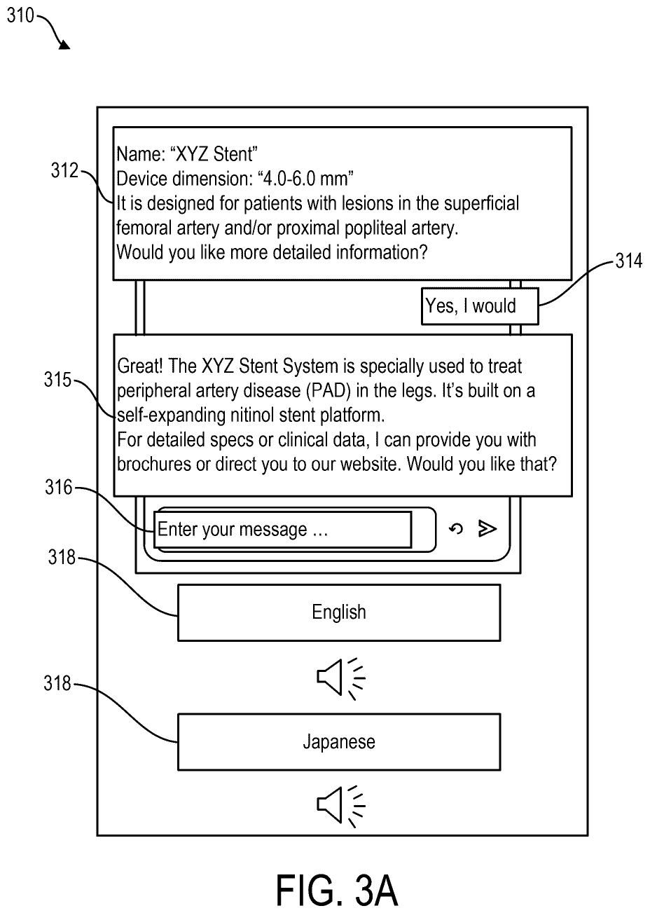

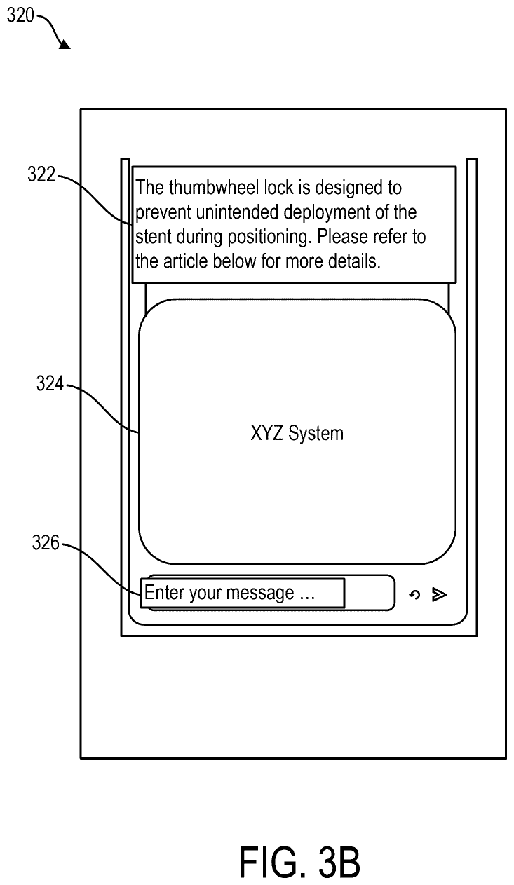

FIG. 3A is an illustrative example of a user interface 310, in accordance with embodiments of the subject matter of the disclosure. In some embodiments, the user interface 310 displays a landing page 312 showing one or more device parameters of the medical device (e.g., a name “XYZ”, a device dimension “4.0-6.0 mm”, an intended use “it is designed for patients with lesions in the superficial femoral artery and/or proximal popliteal artery”, and the like). In some embodiments, the user interface 310 also receives a user response 314 to the displayed landing page 312. In response to the user response 314, the user interface 310 provides corresponding product information 315 of the medical device.

According to some embodiments, the computing device 222 can receive a user query related to the representation of the medical device. In the depicted embodiment of FIG. 2, in certain embodiments, an input/output device 203 (e.g., a microphone of a mobile device) can receive a user query or user response in a vocalized natural language (e.g., English, Japanese, Spanish, and the like). In an embodiment, the user query includes a combination of a vocalized query and a text query. For example, the user interface 310 of FIG. 3A includes a text input field 316 to receive a user text query.

In some embodiments, the computing device 222 further includes a speech-to-text (STT) engine 206 configured to convert the vocalized language captured through an audio input into texts. The STT engine 206 includes, for example, an STT application programming interface (API).

In an embodiment, the STT engine 206 can receive a user vocalized query and apply a multilingual speech recognition model to identify a language of the user vocalized query. In some embodiments, the multilingual speech recognition model can be customized and/or trained for specific use cases of medical devices to accurately transcribe vocalized words into text, including support for various languages and dialects.

In an embodiment, the user interface (UI) engine 204 solicitates a user to indicate or confirm one or more preferred languages. In some embodiments, the user interface 310 of FIG. 3A includes one or more selectable items 318 which allow the user to make choices or provide input to indicate the preferred languages. The one or more selectable items 318 can include, for example, a dropdown menu, a radio button, a checkbox, a toggle switch, and the like.

In some embodiments, the UI engine 204 can provide various user interaction modes. In an example, the UI engine 204 can apply gesture recognition processes to allow users to interact with the system 200 through hand or body gestures, which can be more intuitive in a surgical or sterile environment. In an example, the UI engine 204 can allow the system 200 to recognize and respond to user's vocalized commands/instructions directly, to facilitate a hands-free operation which may be crucial in certain medical settings. In some embodiments, the UI engine 204 can provide multi-modal feedback to users. In an example, the UI engine 204 can allow the application of a wearable device to provide haptic feedback to users as part of an instructional guidance. For example, the haptic feedback can be provided to guide a physician's movements during a process of device setup or operation. In an example, the UI engine 204 can integrate advanced visual (e.g., LED indicators, embedded screens) and auditory cues (e.g., varying tones or alerts) that can guide the usage of medical devices in a more intuitive way.

In some embodiments, the server 224 includes a query engine 210 configured to receive the user query from the UI engine 204 and/or the STT engine 206 and generate a query response by applying a machine learning (ML) model 212 to the received user query and a data repository 214 including information associated with the medical device.

In some embodiments, the query engine 210 includes a prompt generator engine 216 configured to generate a device prompt based at least in part on the received user query and the received one or more device parameters of the medical device. In an embodiment, the prompt generator engine 216 can preprocess the received user query by, for example, removing any noise or irrelevant information that may interfere with interpretation by the machine learning model. In some embodiments, the prompt generator engine 216 can apply natural language processing (NLP) and/or natural language understanding (NLU) techniques or related models to extract key information from the user query to determine the task or intent behind the user's query. Based on the identified task or intent, the prompt generator engine 216 can formulate a prompt template that provides the necessary context and structure for the ML model 212 to generate a query response.

In some embodiments, the query engine 210 can access to an electronic health records (EHR) system to retrieve patient-specific data to tailor the query response or instructions based on the patient's medical history, current condition, and other personalized data. In some embodiments, the query engine 210 can integrate suitable mechanisms for data security and traceability. For example, a blockchain based framework can be integrated into the system 200 to ensure the data integrity and traceability (e.g., data related to user interactions with the instruction system), which can be crucial for compliance and safety in certain medical environments.

In some embodiments, the computing device 222 further includes a text-to-speech (TTS) engine 208 configured to receive a query response generated by the query engine 210 and convert at least a portion of the query response to a voice response in the identified language before delivering the query response. For example, the TTS engine 208 can convert text information of the query response to a voice response in the identified language and deliver the voice response to the user via the input/output device 203 (e.g., an audio device such as a speaker).

FIG. 3B is an illustrative example of a user interface 320, in accordance with embodiments of the subject matter of the disclosure. In some embodiments, the user interface 320 displays text information 322 of a query response, a link to an article 324, and a text input field 326 to receive a user text query. In an embodiment, the TTS engine 208 can convert the text information 322 of the query response to a voice response in the identified language and deliver the voice response to the user. In an embodiment, the TTS engine 208 can convert the text content of the article 324 in response to the user's instruction.

In the embodiment depicted in FIG. 2, according to some embodiments, the system 200 can include one or more computing devices 222 to implement the product information engine 202, the UI engine 204, the STT engine 206, and the TTS engine 208, and one or more servers 224 to include the data repository 214, and the query engine 210. It is to be understood that any one of the functional block components 202, 204, 206, 208, 210 and 214, singularly or in combination, can be realized by one or more computing devices, one or more servers, and a combination of one or more computing devices and servers. It is also to be understood that such functional blocks may be realized by any number of hardware and/or software components configured to perform the specified functions. One or more components of the system 200 are optional and/or can be modified by one or more components of other embodiments described herein. Additionally, one or more components of other embodiments described herein may be added to the system 200.

In some embodiments, each of the computing devices 222 and the servers 224 can be any suitable computing device or combination of devices, such as a desktop computer, a mobile computing device (e.g., a laptop computer, a smartphone, a tablet computer, a wearable computer, and the like), a server computer, a virtual machine being executed by a physical computing device, a web server, and the like. In some embodiments, each of the computing devices 222 and the servers 224 can include a communication system to communicate data with each other over the communication network 228.

The communication network 228 can be any suitable communication network or combination of communication networks. In some embodiments, communication network 228 can include a Wi-Fi network (which can include one or more wireless routers, one or more switches, etc.), a peer-to-peer network (e.g., a Bluetooth network), a cellular network (e.g., a 3G network, a 4G network, a 5G network, etc., complying with any suitable standard), a wired network, and the like. In some examples, communication network 228 can be a local area network (LAN), a wide area network (WAN), a public network (e.g., the Internet), a private or semi-private network (e.g., a corporate or university intranet), any other suitable type of network, or any suitable combination of networks. Communication links (arrows) shown in FIG. 2 can each be any suitable communications link or combination of communication links, such as wired links, fiber optics links, Wi-Fi links, Bluetooth links, cellular links, and the like.

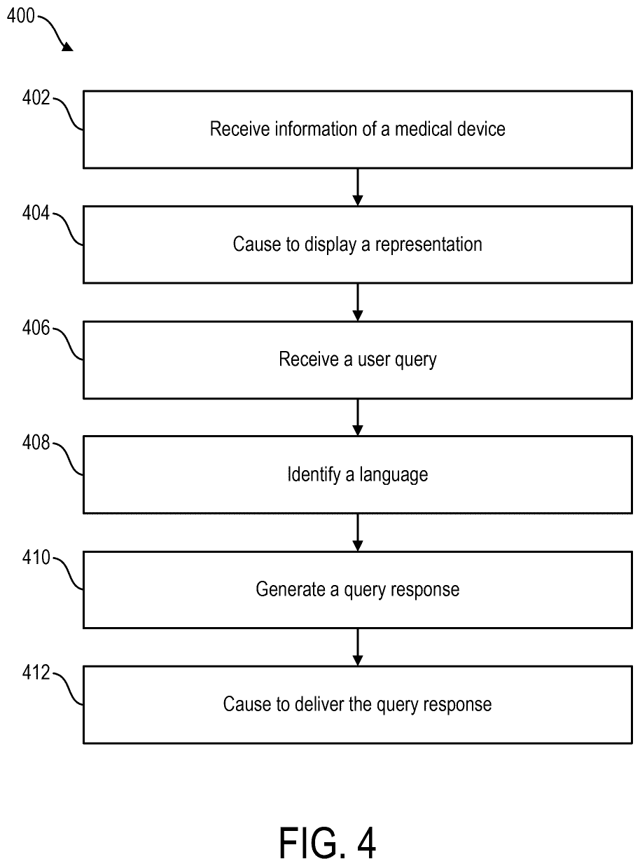

FIG. 4 is an example flow diagram depicting an illustrative method 400 of providing multi-language, speech-to-speech (STS) interactive instructions for a medical device, in accordance with some embodiments of the present disclosure. Aspects of embodiments of the method 400 can be performed, for example, by an IFU system or platform describe herein (e.g., the system 200 depicted in FIG. 2). According to some embodiments, the system (e.g., the system 200) includes one or more computing devices or servers which can be relied on to perform any of a variety of different tasks in the method 400. Further, some of the different tasks may be executed using automated systems, such as models that are trained using artificial intelligence (AI) or machine learning (ML). One or more steps or blocks of method 400 are optional and/or can be modified by one or more steps of other embodiments described herein. Additionally, one or more steps or blocks of other embodiments described herein may be added to the method 400.

At process 402 (“Receive information of a medical device”), in some embodiments, the system 200 receives information associated with the medical device. In some embodiments, the received information associated with the medical device can include, for example, one or more of (i) a quick response (QR) code associated with the medical device, (ii) a Uniform Resource Locator (URL) associated with the medical device, (iii) a smart tag associated with the medical device, (iv) a near-field-communication (NFC) tag, (v) a radio-frequency identification (RFID) tag, and (vi) a product image.

In an embodiment, a user (e.g., a physician) can use a mobile device to scan a unique QR code provided on a product packaging of the medical device. The product information engine 202 of the system 200 can receive and process the unique QR code to identify one or more device parameters of the medical device.

In an embodiment, shortened URLs can be printed on a product packaging of the medical device. A user (e.g., a physician) may use a mobile device to scan the shortened URLs or manually type into the mobile device. The product information engine 202 of the system 200 can receive and process the input of the shortened URLs to identify one or more device parameters of the medical device.

In an embodiment, a smart tag, including a near-field-communication (NFC) tags can be provided (e.g., embedded in or attached to) on a product packaging of the medical device. In some embodiments, a user (e.g., a physician) can tap an NFC-enabled device (e.g., a smartphone) on the smart tag to obtain smart tag information associated with the medical device. The product information engine 202 of the system 200 can receive and process the smart tag information to identify one or more device parameters of the medical device. The method 400 then proceeds to process 404.

At process 404 (“Cause to display a representation”), in certain embodiments, the user interface engine (e.g., the UI engine 204 of the system 200) generates a representation of the medical device associated with the one or more device parameters of the medical device and causes an output device to display the representation. In some embodiments, the representation can include an overview of the medical device including, for example, name, serial number, model number, relevant identifying information, and the like. The representation can be displayed, via a user interface, on a landing page for the medical device. The method 400 then proceeds to process 406.

At process 406 (“Receive a user query”), according to some embodiments, the user interface engine (e.g., the UI engine 204 of the system 200) receives a user query related to the representation of the medical device. In an embodiment, the user query may be received by an input device (e.g., a microphone) in a vocalized natural language. In an embodiment, the user query can include a text query, and/or a combination of a vocalized query and a text query. The method 400 then proceeds to process 408.

At process 408 (“Identify a language”), according to some embodiments, the speech-to-text (STT) engine (e.g., the speech-to-text (STT) engine 206 of the system 200) identifies a language of the user query. In an embodiment, the speech-to-text (STT) engine 206 can include an STT application programming interface (API) which can convert the vocalized language captured through an audio input into texts. In an embodiment, the STT engine 206 of the system 200 can apply a multilingual speech recognition and synthesis model to identify the language of the vocalized query. In some embodiments, the multilingual speech recognition and synthesis model can be customized and/or trained for specific use cases of medical devices to accurately transcribe vocalized words into text, including support for various languages and dialects. In an embodiment, the UI engine 204 can receive a user's indication of preferred language, and/or a user text query to identify the language of the user query. The method 400 then proceeds to process 410.

At process 410 (“Generate a query response”), according to some embodiments, the query engine (e.g., the query engine 210 of the system 200) generates a query response by applying a machine learning model to the received user query and the data repository 214 including information associated with the medical device. The query response can be generated in the identified language.

In an embodiment, the prompt generator engine (e.g., the prompt generator engine 216 of the query engine 210) can generate a device prompt based at least in part on the user query and the one or more device parameters. The query response can then be generated by applying a machine learning model to the device prompt and the data repository 214 including the information associated with the medical device.

In an embodiment, the device prompt can be generated by first preprocessing the user query including, for example, removing any noise or irrelevant information that may interfere with interpretation by the machine learning model. In some embodiments, natural language processing (NLP) techniques or related models can be applied to extract key information from the user query to determine the task or intent behind the user's query. Based on the identified task or intent, the prompt generator engine 216 can formulate a prompt template that provides the necessary context and structure for the machine learning model to generate a query response. In one example, the received use query is “how to use this device A?” The corresponding device prompt may be, for example, “what are the steps of operation device A in model XXX in the next YYY period?”

In an embodiment, the query engine (e.g., the query engine 210 of the system 200) can determine whether the user query meets one or more criteria. For example, a user query may be too simplified, or too confusing, and the prompt generator engine 216 can determine that a suitable device prompt cannot be generated. In some embodiments, when the user query does not meet the one or more criteria, the query engine 210 of the system 200 can generate a query response including contextual information to solicitate the user's clarification. The contextual information can include, for example, a clarification question, an example, a suggestion, and the like. With the user's clarification, the prompt generator engine 216 of the query engine 210 can generate the corresponding device prompt as an input to the machine learning model.

In an embodiment, the machine learning model to be applied by the query engine 210 includes a generative AI (artificial intelligence) model such as, for example, a generative large language model (LLM). It is to be understood that the model may be another type of machine learning model as will be recognized by those of ordinary skill in the art.

In an embodiment, the generation of a query response by applying the machine learning model can be controlled (e.g., modified, allowed, not allowed, etc.) by adjusting one or more content generation parameter of the model to meet one or more query response criteria. In some embodiments, constraints or limitations can be implemented on the model's output to prevent the model from generating responses that go beyond the scope or boundaries of the data repository. For example, a content generation parameter (e.g., a creativity parameter) of a machine learning model can be adjusted between a lower level (e.g., 0) and an upper level (e.g., 1) to control a balance between generating diverse or novel responses (creativity) and ensuring the query responses remain relevant and coherent with the information in the data repository (coherency).

In an embodiment, the query engine (e.g., the query engine 210 of the system 200) can apply a machine learning model with a content generation parameter to generate a query response based at least in part on the content generation parameter. In some embodiments, when the content generation parameter is a first content generation parameter, the query response is a first query response. When the content generation parameter is a second content generation parameter, the query response is a second query response. In some embodiments, when the first content generation parameter is different from the second content generation parameter, the first query response can be different from the second query response. The first query response and the second query response can correspond to the same device content or information in the data repository 214 associated with the medical device. In some examples, when the first content generation parameter (e.g., a creativity parameter at a higher level) is greater than the second content generation parameter (e.g., a creativity parameter at a lower level), the first difference between the first query response and the device content can be greater than the second difference between the second query response and the device content.

In an embodiment, constraints or limitations can be implemented to ensure that the difference between a query response and the corresponding device content in the data repository is not greater than a threshold level. For example, a content generation parameter (e.g., a creativity parameter) can be constrained below a certain level to ensure the coherency.

In an embodiment, regularization techniques can be incorporated into a model architecture or training process of the machine learning model to ensure that the difference between a query response and the corresponding device content in the data repository is not greater than a predetermined threshold level. In some embodiments, a training process of the machine learning model may penalize or discourage the model from generating query responses that deviate too far from the corresponding device content in the data repository 214.

In an embodiment, when the query engine (e.g., the query engine 210 of the system 200) determines that no corresponding information of the medical device can be retrieved from the data repository to respond to the user query, the query engine can generate a query response including contextual information including, for example, contact information for the manufacturer or distributor, customer support and service resources, and the like. In an embodiment, the query engine 210 of the system 200 can generate a query response simply saying, “I do not know.” The method 400 then proceeds to process 412.

At process 412 (“Cause to deliver the query response”), according to some embodiments, the user interface engine (e.g., the UI engine 204 of the system 200) causes to deliver the generated query response in the identified language. In an embodiment, the user interface engine (e.g., the UI engine 204 of the system 200) can receive the generated query response, generate and present a representation of the generated query response in the identified language. The representation can include, for example, text information, an image or graphics, an interactive element (e.g., a button, a dropdown menu, a slider, etc.) to allow the user to further interact with the UI engine 204, a link to text, audio, or video data stored in the data repository 214 or hosted on an external media platform.

In an embodiment, the TTS engine 208 of the system 200 can receive and convert at least a portion of the query response to a voice response in the identified language before delivering the query response. For example, the text information of the query response can be converted to a voice response in the identified language and delivered to the user via an audio device (e.g., a speaker).

In some embodiments, after the delivery of the query response in the identified language, the system 200 waits for a user response to the delivered query response or a new or updated user query, to continue the interaction between the user and the system 200.

In an embodiment, interaction data associated with the interactions between a user and the system 200 can be logged or stored in a memory such as in the data repository 214. The interaction data can include, for example, a user survey, a corresponding device prompt, a corresponding survey response, and the like. In some embodiments, the interaction data can be utilized to monitor common user queries and to identify potential improvements for the medical device and/or instructions for the medical device.

In an embodiment, the logged or stored historical interaction data can be used as feedback to improve the performance of the query engine 210 by applying the ML model 212. In some embodiments, the historical interaction data can be used to analyze the performance of a machine learning model applied by the query engine 210 on previous tasks or scenarios to identify shortcomings. Based on the analysis of historical data, adjustments can be made to the query engine 210 regarding, for example, the generation of a device prompt, the model architecture, features, or algorithms, and the like to address the identified shortcomings and improve performance.

In some embodiments, the query engine 210 can apply the ML model 212 to provide predictive analytics based on historical user data. For example, the query engine 210 can predict failures or required maintenance for medical devices based at least in part on the usage patterns and interaction data collected by the system 200.

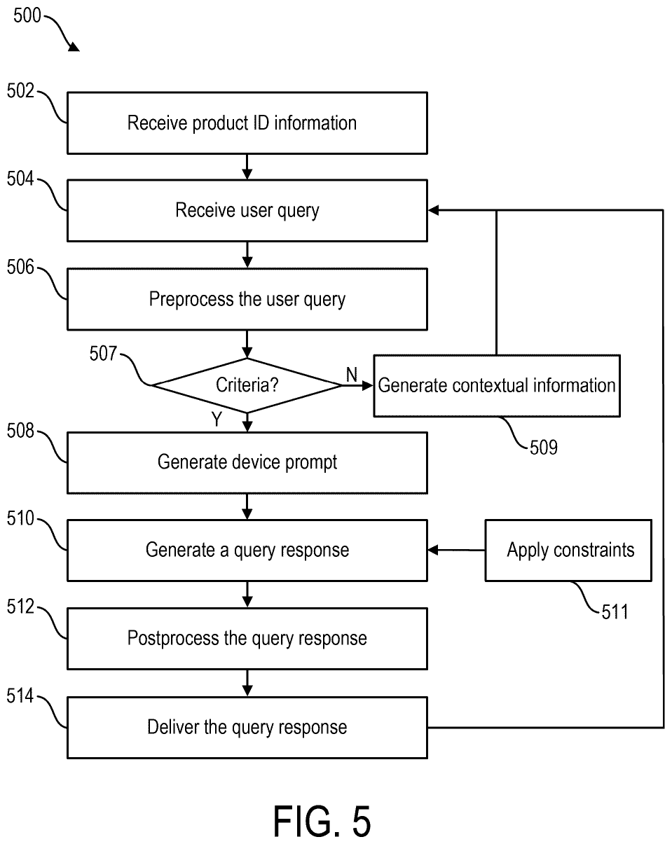

FIG. 5 is an example flow diagram 500 depicting an illustrative method of providing multi-language, speech-to-speech interactive instructions for a medical device, in accordance with some embodiments of the present disclosure. Aspects of embodiments of the method 500 may be performed, for example, by an IFU system or platform describe herein (e.g., the system 200 depicted in FIG. 2). The system 200 includes one or more computing devices or servers which may be relied on to perform any of a variety of different tasks in the method 500. Further, some of the different tasks may be executed using automated systems, such as models that are trained using artificial intelligence (AI) or machine learning (ML). One or more steps or blocks of method 500 are optional and/or can be modified by one or more steps of other embodiments described herein. Additionally, one or more steps or blocks of other embodiments described herein may be added to the method 500.

At process 502 (“Receive product ID information”), according to some embodiments, the user interface engine (e.g., the UI engine 204 of the system 200) receives identification information of the medical device. The received identification information associated with the medical device can include, for example, one or more of (i) a quick response (QR) code associated with the medical device, (ii) a Uniform Resource Locator (URL) associated with the medical device, (iii) a smart tag associated with the medical device, (iv) a near-field-communication (NFC) tag, (v) a radio-frequency identification (RFID) tag, and (vi) a product image. In an embodiment, a user (e.g., a physician) may use a mobile device to detect (e.g., scan an image) a unique QR code provided on a product packaging of the medical device. The system 200 can receive and process the unique QR code to identify one or more device parameters of the medical device associated with the identification information of the medical device. The method 500 then proceeds to process 504.

At process 504 (“Receive a user query”), according to some embodiments, the user interface engine (e.g., the UI engine 204 of the system 200) receives a user query related to the identified medical device. In an embodiment, the user query may be received by an input device (e.g., a microphone) in a vocalized natural language. In an embodiment, the user query may include a combination of a vocalized query and a text query. The method 500 then proceeds to process 506.

At process 506 (“Preprocess the user query”), according to some embodiments, the system 200 preprocesses the user query to identify a language of the user query. In an embodiment, the speech-to-text (STT) engine 206 can include, for example, an STT application programming interface (API), which can convert the vocalized language captured through an audio input device into texts. In an embodiment, the STT engine 206 can apply a multilingual speech recognition model to identify the language of the vocalized query. In some embodiments, the multilingual speech recognition model can be customized and/or trained for specific use cases of medical devices to accurately transcribe vocalized words into text, including support for various languages and dialects. In an embodiment, the query engine 210 may preprocess the user query including, for example, removing any noise or irrelevant information that may interfere with interpretation by the machine learning model. The method 500 then proceeds to process 507.

At process 507 (“Criteria?”), according to some embodiments, the query engine (e.g., the query engine 210 of the system 200) can determine whether the user query meets one or more criteria. In an example, the query engine 210 can determine whether a user query is too simplified, or too confusing such that a suitable device prompt cannot be generated. In some examples, the query engine can generate a prompt and/or provide the prompt to the user to provide additional information if the one or more criteria are not met. For example, the query engine may ask the user, “is this a Company A product?”. In an example, the query engine can determine whether a user query pertains to certain products of a certain manufacturer. In some embodiments, the query engine 210 can apply natural language processing (NLP) techniques or related models to extract key information from the user query to determine whether the user query meets one or more predetermined criteria. In some embodiments, when the query engine 210 of the system 200 determines that user query meets the one or more predetermined criteria, the method 500 proceeds to process 508. When the query engine 210 of the system 200 determines that user query does not meet the one or more predetermined criteria, the method 500 proceeds to process 509.

At process 508 (“Generate device prompt”), when the query engine 210 of the system 200 determines that the user query meets the one or more predetermined criteria, the prompt generator engine (e.g., the prompt generator engine 216 of the system 200) generates a device prompt based at least in part on the user query and the one or more device parameters of the medical device. The method 500 proceeds to process 510.

At process 509 (“Generate contextual information”), according to some embodiments, when the query engine 210 of the system 200 determines that the user query does not meet the one or more predetermined criteria, the prompt generator engine (e.g., the prompt generator engine 216 of the system 200) generates contextual information to solicitate the user's clarification. The contextual information can include, for example, a clarification question, an example, a suggestion, and the like. The method 500 then proceeds back to process 504 to receive an updated user query.

At process 510 (“Generate a query response”), the query engine (e.g., the query engine 210 of the system 200) generates a query response by applying a machine learning model to the device prompt and a data repository including information associated with the medical device.

In an embodiment, the machine learning model may include a generative large language model (LLM). It is to be understood that the model may be another type of machine learning model as will be recognized by those of ordinary skill in the art.

In an embodiment, the query engine 210 of the system 200 can apply constraints or limitations at process 511 (“Apply constraints”) to the model's output to prevent the model from generating responses that go beyond the scope or boundaries of the data repository 214. For example, the data repository 214 may store contents of IFUs that are regulated. When the query engine 210 determines that a relevant answer cannot be provided by the IFUs, the query engine 210 can generate a suitable query response (e.g., “I do not know,” “please contact the representative,” “please rephrase or restate your question,” and the like), instead of making up answers that go beyond the scope of the contents contained in the regulated IFUs.

In some embodiments, a content generation parameter (e.g., a creativity parameter) of a machine learning model may be adjusted between a lower level (e.g., 0) and an upper level (e.g., 1) to control a balance between generating diverse or novel responses (creativity) and ensuring the query responses remain relevant and coherent with the information in the data repository (coherency). In an embodiment, constraints or limitations can be implemented to ensure that the difference between a query response and the corresponding device content in the data repository is not greater than a threshold level. For example, a content generation parameter (e.g., a creativity parameter) can be constrained below certain level to control the difference.

In an embodiment, constraints or limitations at process 511 (“Apply constraints”) can be applied by incorporating regularization techniques into a model architecture or training process of the machine learning model to ensure that the difference between a query response and the corresponding device content in the data repository is not greater than a predetermined threshold level. In some embodiments, a training process of the machine learning model may penalize or discourage the model from generating query responses that deviate too far from the corresponding device content in the data repository. The method 500 then proceeds to process 512.

At process 512 (“Postprocess the query response”), according to some embodiments, the system 200 postprocesses the generated query response. The query engine 210 can process the query response to be delivered in the identified language. In an embodiment, the query engine 210 can process the generated query response to determine one or more suitable formats to deliver the query response. In some embodiments, the query engine 210 can generate a visual representation of the query response. The representation can include, for example, text information, an image or graphics, an interactive element (e.g., a button, a dropdown menu, a slider, etc.) to allow the user to further interact with the UI engine 204, a link to text, audio, or video data stored in the data repository 214 or hosted on an external media platform. In some embodiments, the query engine 210 can also extract text information from the query response, which can be converted to a voice response by the TTS engine 208. The method 500 then proceeds to process 514.

At process 514 (“Deliver the query response”), the user interface engine (e.g., the UI engine 204 of the system 200) causes to deliver the generated query response in the identified language. In an embodiment, the UI engine 204 can receive the processed query response from the query engine 210 and present the visual representation of the query response. In some embodiments, the TTS engine 208 can receive the processed query response from the query engine 210 and convert at least a portion of the query response to a voice response in the identified language before delivering the query response. For example, the text information of the query response can be converted to a voice response in the identified language and delivered to the user via an audio device (e.g., a speaker). The method 500 then proceeds back to process 502 to receive a new or updated user query.

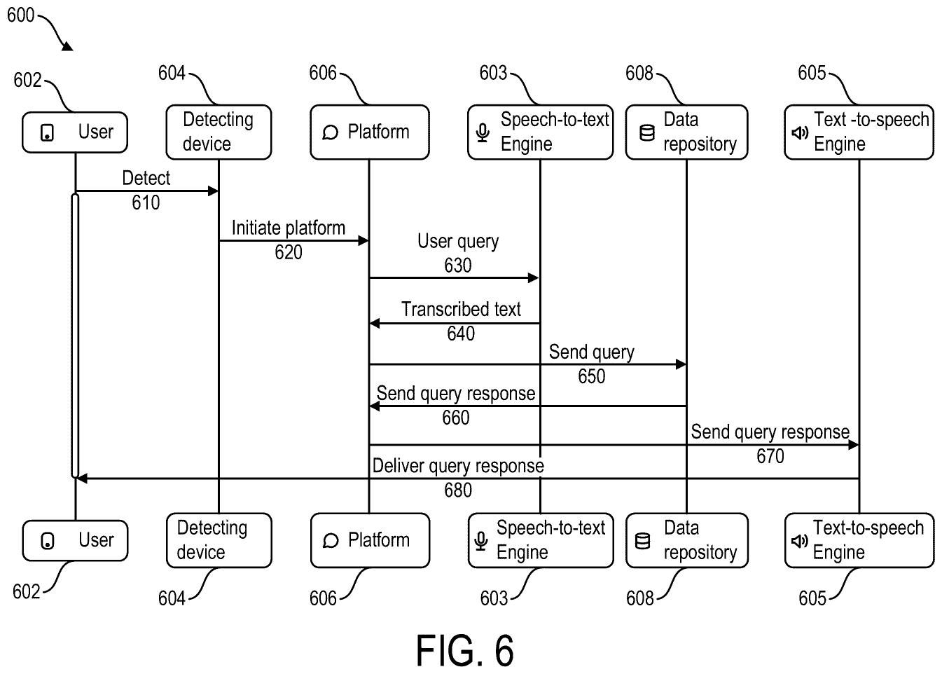

FIG. 6 is an example block diagram 600 depicting an illustrative method of providing multi-language, speech-to-speech (STS) interactive instructions for a user to use a medical device, in accordance with some embodiments of the present disclosure. At process 610, a user 602 (e.g., a physician) can detect a product label (e.g., a QR code) on a packaging of the medical device by using a detecting device 604 (e.g., a smartphone, a tablet, a QR code reader, an NFC-enabled device, and the like) to obtain product identification information associated with the medical device. It is to be understood that the identification information can be obtained by any suitable mechanisms described herein.

At process 620, the obtained product identification information can initiate an IFU platform or system 606. For example, the detecting device 604 can detect the QR code and decode its contents. The platform or system 606 receives the decoded information and displays the decoded information via a user interface. In an embodiment, the decoded information can include a URL (e.g., a web link) directed to a landing page introducing the medical device, for example, containing one or more device parameters of the medical device.

At process 630, an input audio device (e.g., a microphone of a mobile device) receives a voice or vocalized query from the user 602 in certain vocalized language and send the received audio signal to a speech-to-text API 603.

At process 640, the speech-to-text API 603 converts the received audio signal to a transcribed text. In an embodiment, the speech-to-text API 603 can identify the language of the user query.

At process 650, the platform or system 606 sends the user query including the transcribed text to a query engine to generate a query response by applying a machine learning model to the user query and a data repository 608 including information associated with the medical device.

At process 660, the query engine sends the generated query response to the platform or system 606.

At process 670, the platform or system 606 sends the received query response to a text-to-speech API 605 to convert at least a portion of the query response to a voice response in the identified language of the user query.

At process 680, the text-to-speech API 605 sends the voice response in the identified language to an output audio device (e.g., a speaker) to deliver to the user 602.

After the user 602 receives the query response in the identified language, the user 602 can provide a user response or a new or updated user query to continue the interaction with the platform or system 606. In this manner, the platform or system 606 can provide step-by-step voice instructions and to respond to the user vocalized queries related to the medical device including, for example, device functions, maintenance, safety precautions, troubleshooting, and the like.

The present disclosure describes systems and methods for providing interactive instructions for a medical apparatus or device. More specifically, some embodiments of the present disclosure relate to systems and methods for providing multi-language, speech-to-speech interactive instructions for a medical apparatus or device. It is to be understood that the systems and methods described herein can have various applications including, for example, various patient guides/instructions to patients (e.g., for products that patients can take home), quick reference guides, customer training manuals, aids for field sales representatives, internal training for field sales representatives, compliance charts, in particular, for medical electrical equipment (MEE) user manuals and quick reference guides which have usually a large size (e.g., more than 100 pages).

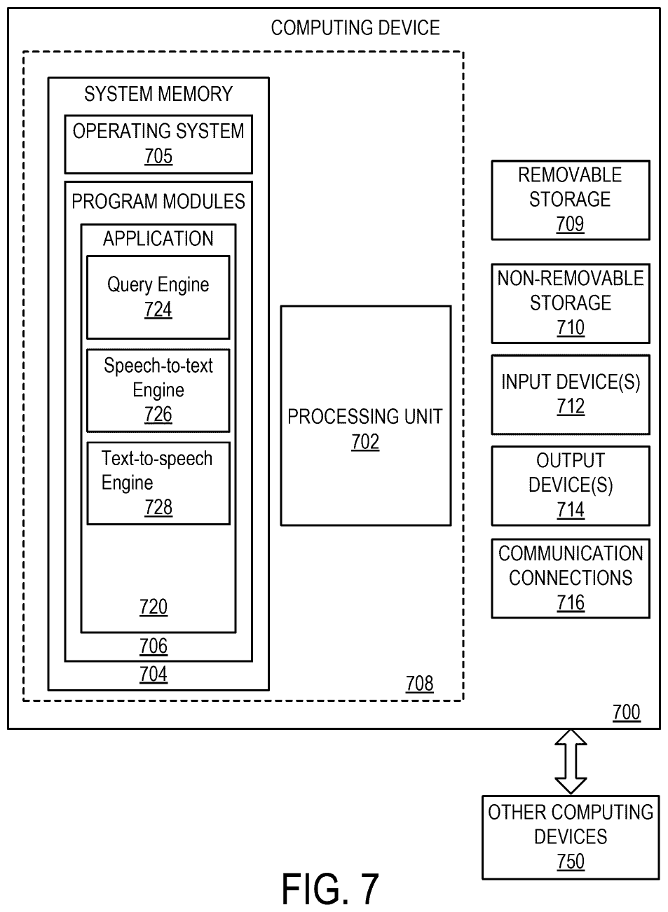

FIG. 7 is a simplified block diagram of a computing device 700, with which aspects of the present disclosure may be practiced. The computing device components described below may be suitable for the computing devices described above, including the computing device 222 and/or the server 224 in FIG. 2. The computing device 700 may include at least one processing unit 702 and a system memory 704. Depending on the configuration and type of computing device, the system memory 704 may include, for example, volatile storage (e.g., random access memory), non-volatile storage (e.g., read-only memory), flash memory, or any combination of such memories.

The system memory 704 may include an operating system 705 and one or more program modules 706 suitable for running software application 720, such as one or more components supported by the systems described herein. As examples, system memory 704 may store query engine or processor 724, speech-to-text engine or processor 726, and/or text-to-speech engine or processor 728. The operating system 705, for example, may be suitable for controlling the operation of the computing device 700.

A basic configuration is illustrated in FIG. 7 by those components within a dashed line 708. The computing device 700 may have additional features or functionality. For example, the computing device 700 may also include additional data storage devices (removable and/or non-removable) such as, for example, magnetic disks, optical disks, or tape. Such additional storage is illustrated in FIG. 7 by a removable storage device 709 and a non-removable storage device 710.

As stated above, a number of program modules and data files may be stored in the system memory 704. While executing on the processing unit 702, the program modules 706 (e.g., application 720) may perform processes including, but not limited to, the aspects, as described herein. Other program modules that may be used in accordance with aspects of the present disclosure may include electronic mail and contacts applications, word processing applications, spreadsheet applications, database applications, slide presentation applications, drawing or computer-aided application programs, and the like.

Furthermore, aspects of the disclosure may be practiced in an electrical circuit comprising discrete electronic elements, packaged or integrated electronic chips containing logic gates, a circuit utilizing a microprocessor, or on a single chip containing electronic elements or microprocessors. For example, aspects of the disclosure may be practiced via a system-on-a-chip (SOC) where each or many of the components illustrated in FIG. 7 may be integrated onto a single integrated circuit. Such an SOC device may include one or more processing units, graphics units, communications units, system virtualization units and various application functionality all of which are integrated (or “burned”) onto the chip substrate as a single integrated circuit. When operating via an SOC, the functionality, described herein, with respect to the capability of client to switch protocols may be operated via application-specific logic integrated with other components of the computing device 700 on the single integrated circuit (chip). Some aspects of the disclosure may also be practiced using other technologies capable of performing logical operations such as, for example, AND, OR, and NOT, including but not limited to mechanical, optical, fluidic, and quantum technologies. In addition, some aspects of the disclosure may be practiced within a general-purpose computer or in any other circuits or systems.

The computing device 700 may also have one or more input device(s) 712 such as a keyboard, a mouse, a pen, a sound or voice input device, a touch or swipe input device, and the like. Output device(s) 714 such as a display, speakers, a printer, etc. may also be included. The aforementioned devices are examples and others may be used. The computing device 700 may include one or more communication connections 716 allowing communications with other computing devices 750. Examples of suitable communication connections 716 include, but are not limited to, radio frequency (RF) transmitter, receiver, and/or transceiver circuitry; universal serial bus (USB), parallel, and/or serial ports.

The term computer readable media as used herein may include computer storage media. Computer storage media may include volatile and nonvolatile, removable and non-removable media implemented in any method or technology for storage of information, such as computer readable instructions, data structures, or program modules. The system memory 704, the removable storage device 709, and the non-removable storage device 710 are all computer storage media examples (e.g., memory storage). Computer storage media may include RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other article of manufacture which can be used to store information and which can be accessed by the computing device 700. Any such computer storage media may be part of the computing device 700. Computer storage media does not include a carrier wave or other propagated or modulated data signal.

Communication media may be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media.

Various modifications and additions can be made to the exemplary embodiments discussed without departing from the scope of the present disclosure. For example, while the embodiments described above refer to particular features, the scope of the present disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A method of providing interactive instructions for a medical device, the method comprising:
receiving information associated with the medical device;
identifying one or more device parameters of the medical device;
causing to display a representation of the medical device associated with the one or more device parameters of the medical device;
receiving a user query related to the representation of the medical device;
identifying a language of the user query;
generating a device prompt based at least in part on the user query and the one or more device parameters;
generating a query response by applying a machine learning model to the device prompt and a data repository including information associated with the medical device, the query response being in the identified language; and
causing to deliver the query response in the identified language.

2. The method of claim 1, wherein the receiving information associated with the medical device comprises receiving at least one selected from a group consisting of a quick response (QR) code, a Uniform Resource Locator (URL), a near-field-communication (NFC) tag, a radio-frequency identification (RFID) tag, and a product image.

3. The method of claim 1, further comprising retrieving the one or more device parameters of the medical device from the data repository.

4. The method of claim 1, wherein the receiving a user query further comprises receiving a vocalized query.

5. The method of claim 4, further comprising converting the vocalized query to a transcribed text.

6. The method of claim 1, further comprising determining whether the user query meets one or more criteria.

7. The method of claim 6, wherein when the user query does not meet the one or more criteria, the generating a query response further comprises generating contextual information of the medical device from the data repository.

8. The method of claim 1, wherein the applying a machine learning model comprises:
generating an improved query response for the user query using the user query and the query response.

9. The method of claim 1, wherein the generating a query response further comprises providing one or more query response constraints.

10. The method of claim 1, wherein the generating a query response further comprises adjusting a content generation parameter.

11. The method of claim 10, wherein the query response generated based on the content generation parameter corresponds to a device content in the data repository, and the content generation parameter is adjusted such that a difference between the query response and the device content in the data repository is not greater than a predetermined level.

12. The method of claim 11, wherein the content generation parameter is adjusted to be lower than a predetermined threshold to control a coherency between the query response and the device content in the data repository.

13. The method of claim 1, further comprising converting the query response to a voice response in the identified language before delivering the query response.

14. A system of providing interactive instructions for a medical device, the system comprising:
one or more memories having instructions stored thereon; and
one or more processors configured to execute the instructions and perform operations comprising:
receiving information associated with the medical device;
identifying one or more device parameters of the medical device;
causing to display a representation of the medical device associated with the one or more device parameters of the medical device;
receiving a user query related to the representation of the medical device;
identifying a language of the user query;
generating a device prompt based at least in part on the user query and the one or more device parameters;
generating a query response by applying a machine learning model to the device prompt and a data repository including information associated with the medical device, the query response being in the identified language; and
causing to deliver the query response in the identified language.

15. The system of claim 14, wherein the operations further comprise receiving at least one selected from a group consisting of a quick response (QR) code, a Uniform Resource Locator (URL), a near-field-communication (NFC) tag, a radio-frequency identification (RFID) tag, and a product image.

16. The system of claim 14, wherein the operations further comprise converting a vocalized query to a transcribed text.

17. The system of claim 14, wherein the operations further comprise determining whether the user query meets one or more criteria, and when the user query does not meet the one or more criteria, generating contextual information of the medical device from the data repository.

18. The system of claim 14, wherein the operations further comprise adjusting a content generation parameter to meet one or more query response constraints.

19. The system of claim 18, wherein the query response generated based on the content generation parameter corresponds to a device content in the data repository, and the content generation parameter is adjusted such that a difference between the query response and the device content in the data repository is not greater than a predetermined level.

20. The system of claim 14, wherein the operations further comprise converting the query response to a voice response in the identified language before delivering the query response.
